(19) United States
(12) Patent Application Publication
(10) Pub. No.: US 2025/0025872 A1
(43) Pub. Date: Jan. 23, 2025

(54) SAMPLE COLLECTION DEVICE AND SYSTEM

(71) Applicant: Gregor Diagnostics, Madison, WI (US)

(72) Inventors: Sisi Li, Madison, WI (US); Joel Wipperfurth, Madison, WI (US); Emily Breunig, Madison, WI (US); Tobias Zutz, Madison, WI (US); Mia Shanholtzer, Madison, WI (US); D. Paul Barnes, Madison, WI (US)

(21) Appl. No.: 18/714,999

(22) PCT Filed: Nov. 30, 2022
(86) PCT No.: PCT/US2022/080668

§ 371 Date: May 30, 2024

Related U.S. Application Data
(60) Provisional application No. 63/284,386, filed on Nov. 30, 2021.

(51) Int. Cl. B01L 3/00; A61B 10/00

(52) U.S. Cl. CPC B01L 3/502; A61B 10/0058; B01L 2200/025; B01L 2200/0689; B01L 2200/185; B01L 2300/042; B01L 2300/0681; B01L 2300/069

(57) ABSTRACT

Provided herein are devices, systems, and methods for sample collection. In particular, provided herein are devices, systems, and methods that employ sample collection devices in which the sample is deposited on a capture material and dried via desiccation.

### CROSS-REFERENCE TO RELATED APPLICATIONS

This application claims the benefit of U.S. Provisional Application No. 63/284,386, filed Nov. 30, 2021, the content of which is herein incorporated by reference in its entirety.

### FIELD

Provided herein are devices, systems, methods, and kits for sample collection, storage, and transport.

### BACKGROUND

Increasingly, samples for analysis are often collected at locations external to site of analysis, thereby requiring some method for storage, transportation, and stabilization of the analytes of interest contained within the sample. Liquid samples may be stored and transported in sealed containers under refrigerated or frozen conditions. These methods can encounter many problems, including cost of cooling mechanism, cooling instability during shipment, risk of breakage or leakage of container and refusal by shipping carrier is sample is classified as a potential biohazard. Dried samples can provide greater ease of transport. Due to the wide variety of matrices and drying methods, however, analyte instability during and upon drying and the necessary extraction methods to remove the analyte from the capture matrix can result in a lack of precision and accuracy in downstream analysis.

### SUMMARY

Provided herein are devices, systems, and methods for sample collection, storage, and transport.

In some embodiments, the devices comprise a reservoir configured for collecting and holding a sample and a base comprising one or more sample regions (e.g., sample wells). In some embodiments, the device further comprises a cap.

In some embodiments, the systems comprise a reservoir configured for collecting and holding a sample, a base comprising one or more sample wells, and a cap.

In some embodiments, the cap comprises desiccant. In some embodiments, the desiccant is selected from silica gel, molecular sieve, an aluminosilicate, or a combination thereof.

In some embodiments, the one or more sample wells each comprises a capture material. In some embodiments, the capture material comprises filter paper, cotton linter, or a combination thereof.

In some embodiments, the reservoir is adjustably, fluidly connected each of the one or more sample wells. In some embodiments, a sample passage aperture in a bottom wall of the reservoir is configured to align with the one or more sample wells in the base at a starting position or upon rotation of the reservoir. In some embodiments, the reservoir comprises an inner surface which is tapered to the sample passage aperture.

In some embodiments, the reservoir is detachably connected to the base. In some embodiments, the reservoir comprises catches protruding from a lower face and the base comprises tabs configured to align the reservoir and the base.

In some embodiments, the base is configured to independently attach to the reservoir and the cap.

In some embodiments, the reservoir further comprises a shutoff device to control fluid connection between the sample passage aperture and the sample wells. In some embodiments, the shutoff device comprises an opening to fluidly connect the reservoir and the one or more sample wells.

In some embodiments, the base further comprises a retention ring which functionally integrates with the shutoff device. In select embodiments, the shutoff device comprises a pin which functionally integrates with a slot in the retention ring facilitating rotation and removal of the reservoir from the base.

In some embodiments, the retention ring comprises one or more retention ring apertures configured to align with the shutoff device opening to fluidly connect the reservoir and the one or more sample wells.

In some embodiments, the cap is configured to attach to the base following detachment of reservoir. In some embodiments, the cap is a screw cap.

In some embodiments, the devices and system provided herein further comprise a sample. In some embodiments, the sample comprises a biological fluid (e.g., semen, secretions, and the like). In some embodiments, the sample is in the one or more sample wells. In some embodiments, the sample is dried on the capture material.

In some embodiments, the methods comprise at least one or all of: providing a device or system as disclosed herein, providing a sample in the reservoir, rotating the reservoir to fill each of the one or more sample wells, removing the reservoir, and capping the base. In some embodiments, the methods further comprise breaking a tamper resistant seal. In some embodiments, the methods further comprise removing the seal from the cap prior to capping the base.

In some embodiments, the methods comprise collecting a sample in a device or system described herein, removing the reservoir, and capping the base.

In some embodiments, the methods comprise: collecting a semen sample in a device comprising a plurality of regions (e.g., two, three, four, or more) each having a capture material such that a portion of the semen sample is retained by the capture material in said plurality of regions; and drying the semen sample.

In some embodiments, the methods further comprise sealing the device to enclose the sample. In some embodiments, at least a portion of the drying step occurs following the sealing.

In some embodiments, the methods further comprise shipping the device to a testing location.

In some embodiments, the methods further comprise analyzing the sample after the drying step. In certain embodiments, analyzing comprises conducting a biological assay with sample from obtained from capture material from one or more or all of the regions.

In some embodiments, the methods comprise collecting a semen sample on a plurality of capture materials; drying the semen sample; storing and/or shipping dried semen samples; and conducting a biological assay on sample obtained from one or more of the plurality of capture materials.

Further provided herein are kits comprising in a device or system described herein and one or more of: a disposal bag, a shipping container, and instructions.

### BRIEF DESCRIPTION OF THE DRAWINGS

FIG. 1 shows an exemplary sample collection system comprising a reservoir 200, a base 300, and a cap 400, wherein the reservoir and base are functionally attached.

FIG. 2 is an expanded view of an exemplary device comprising a reservoir 200 and base 300.

FIG. 3 is an expanded view of an exemplary device comprising a base 300 and a cap 400.

FIG. 4 is side view of an exemplary base 300, reservoir 200, and cap 400.

FIG. 5 is schematic showing an exemplary methodology of using an exemplary device and/or system in which the sample is collected in the reservoir and the proctor or user of the device rotates the reservoir to each of the marked sample wells to transfer a portion of the sample from the reservoir to the capture material and then to remove the reservoir completely before applying the cap to the base for sample drying and transport.

### DETAILED DESCRIPTION

The present disclosure provides technology related to devices, systems, and methods for sample collection, storage, and transport. The devices and systems disclosed herein find use in a variety of settings. Particularly, the present disclosure provides devices, systems, and methods for biological sample collection in external locations (e.g., at home or non-healthcare facilities) that are easy to use, accurate, and convenient for external sample procurement, storage and drying of samples, and shipment to testing facilities.

Section headings as used in this section and the entire disclosure herein are merely for organizational purposes and are not intended to be limiting.

### 1) Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “subject” refers to any animal (e.g., a mammal), including, but not limited to, humans, non-human primates, rodents, and the like. Typically, the terms “subject” and “patient” are used interchangeably herein in reference to a human subject. As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “system” denotes a set of components comprising a whole where each component interacts with or is related to at least one other component within the whole.

As used herein, an element of the present technology is “integral” to another element of the present technology when the two elements are manufactured or assembled as a single piece.

As used herein, an element of the present technology is “separate” from another element of the present technology when the two elements are manufactured or provided as separate pieces.

As used herein, the term “analyte” refers to a compound, natural or synthetic, or composition to be detected or measured. An analyte can include, but is not limited to, a drug, a hormone, a protein, a nucleic acid, a carbohydrate, an element, an ion, a small molecule (e.g., a natural or synthetic small molecule), a biomarker. A “biomarker” includes a biological compound, such as a protein and a fragment thereof, a peptide, a polypeptide, a proteoglycan, a glycoprotein, a lipoprotein, a carbohydrate, a lipid, a nucleic acid, an organic on inorganic chemical, a natural polymer, a cell fragment, a microparticle, an exosome, and a small molecule, that is present in a biological sample and that may be isolated from, or measured in the biological sample. Furthermore, a biomarker may be the entire intact molecule, or a portion thereof that may be partially functional or recognized, for example, by an antibody or other specific binding protein. A biomarker may be associated with a given state of a subject, such as a particular stage of disease. In some embodiments, the biomarker is a cancer biomarker (e.g., circulating tumor DNA, protein biomarkers (e.g., prostate specific antigen, alpha-fetoprotein, carcinoembryonic antigen)). A measurable aspect of a biomarker may include, for example, the presence, absence, or concentration of the biomarker in the biological sample from the subject and/or relative changes of any of the measurable aspects compared to a standard (e.g., internal or from a healthy subject). The measurable aspect may also be a ratio of two or more measurable aspects of two or more biomarkers. Biomarker, as used herein, also encompasses a biomarker profile comprising measurable aspects of two or more individual biomarkers. The two or more individual biomarkers may be from the same or different classes of biomarkers such as, for example, a nucleic acid and a carbohydrate, or may measure the same or different measurable aspect such as, for example, absence of one biomarker and concentration of another. A biomarker profile may comprise any number of individual biomarkers or features thereof. In another embodiment, the biomarker profile comprises at least one measurable aspect of at least one internal standard. Methods of identifying and quantifying biomarkers are well known in the art and include histological and molecular methods such as enzyme-linked immunosorbent assays (ELISA) and other immunoassays, gel electrophoresis, protein and DNA arrays, mass spectrometry, colorimetric assays, electrochemical assays, and fluorescence methods.

As used herein, the term “sample” is used in its broadest sense. In one sense, it is meant to include a specimen obtained from any source, including biological and environmental samples. Biological samples may be obtained from animals (including humans) and encompass fluids, solids, tissues, and gases. Such examples are not however to be construed as limiting the sample types. Preferably, a sample is a fluid sample such as a liquid sample. Examples of liquid samples that may be assayed include bodily fluids (e.g., blood, serum, plasma, saliva, urine, ocular fluid, semen, sputum, sweat, tears, and spinal fluid), water samples (e.g., samples of water from oceans, seas, lakes, rivers, and the like), samples from home, municipal, or industrial water sources, runoff water, or sewage samples; and food samples (e.g., milk, beer, juice, or wine). Viscous liquid, semisolid, or solid specimens may be used to create liquid solutions, eluates, suspensions, or extracts that can be samples. For example, throat or genital swabs may be suspended in a liquid solution to make a sample. Samples can include a combination of liquids, solids, gasses, or any combination thereof (e.g., a suspension of lysed or unlysed cells in a buffer or solution). Samples can comprise biological materials, such as cells, microbes, organelles, and biochemical complexes. Liquid samples can be made from solid, semisolid, or highly viscous materials, such as soils, fecal matter, tissues, organs, biological fluids, or other samples that are not fluid in nature. For example, solid or semisolid samples can be mixed with an appropriate solution, such as a buffer, a diluent, and/or extraction buffer. The sample can be macerated, frozen and thawed, or otherwise extracted to form a fluid sample. Residual particulates may be removed or reduced using conventional methods, such as filtration or centrifugation.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. Illustrative embodiments of the invention are shown in the figures. It should be understood that the invention is not limited to these particular examples.

### 2) Devices and Systems

The technology relates to devices and systems for collecting, storing, and/or transporting a sample, including liquid samples as described herein. In some embodiments, the devices comprise a reservoir and a base. The systems comprise a reservoir for collecting and holding a sample, a base comprising one or more sample wells, and a cap comprising a desiccant.

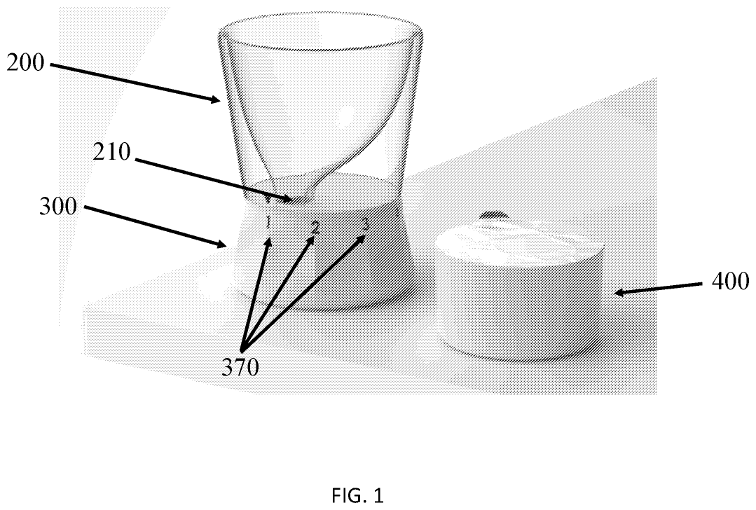

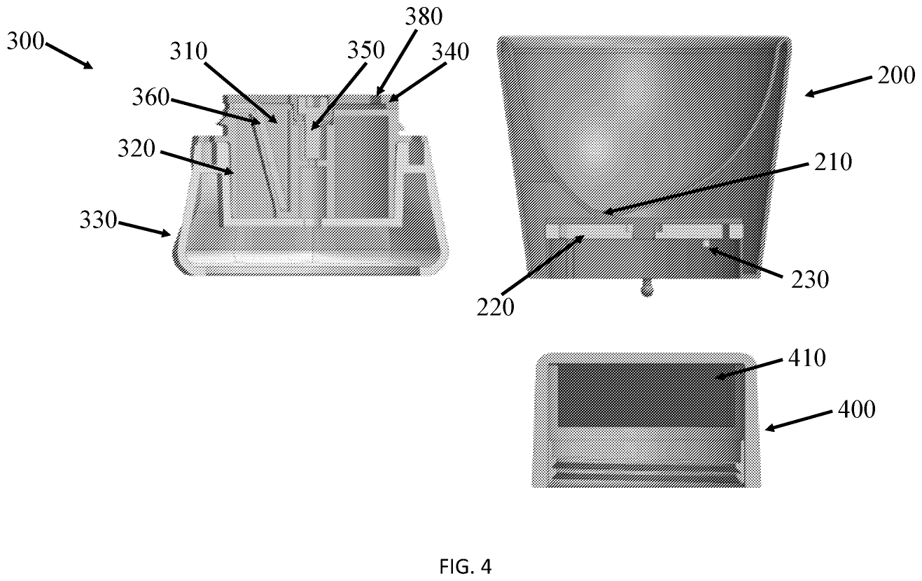

In some embodiments, base 300 is adjustably fluidly connected to reservoir 200 by a sample passage aperture 210 in the bottom of reservoir 200 (FIG. 1). Reservoir 200 is capable of collecting a sample through an opening defined by the upper portion of the reservoir. Embodiments provided herein comprise a reservoir 200, having an inner surface and an outer surface (FIGS. 1 and 4). The outer surface may have a symmetrical shape that is cylindrical, convex, conical, elliptical, square, or rectangular. Preferably, the outer surface is of a shape to facilitate ease of handling and holding.

The inner surface of reservoir 200 may be any shape size or shape necessary to define the appropriate volumetric size for the sample. In some embodiments, the inner surface comprises side walls which are arched to form a cylindrical chamber. In some embodiments, the inner surface comprises four side walls, each perpendicular, substantially perpendicular, and/or essentially perpendicular to the adjacent side wall, such as to for a rectangle or square. In some embodiments, the inner surface is angled or contoured, thereby directing flow of a sample towards reservoir sample passage aperture 210. For example, the inner surface may be contoured to a funnel or funnel-like shape.

The size of reservoir 200 is appropriate to meet or exceed the expected volumetric size of the sample to be contained and/or held within the reservoir. As a lower limit, embodiments provide that the reservoir 210 volume is sufficiently large to transfer an adequate volume of a sample to base 300, considering adhesive forces between the materials of construction and the sample that reduce transfer of the sample to the base. As an upper limit, embodiments provide that the reservoir volume is sufficiently small to prevent the sample from overloading the base 300 due to forces exerted from a sample entering the base 300 when the reservoir is full.

In exemplary embodiments, the reservoir comprises a size to accommodate sample volumes greater than approximately 0.25 mL, 0.5 mL, 1 mL, 2, mL, 3 mL, 4 mL, 5 mL, 6 mL, 7 mL, 8 mL, 9 mL, 10 mL, or more. In some embodiments, the size of the reservoir accommodates sample volumes of 0.25-20 mL, 0.25-15 mL, 0.25-10 mL, 0.25-5 mL, 0.5-20 mL, 0.5-15 mL, 0.5-10 mL, 0.5-5 mL, 0.1-20 mL, 0.1-15 mL, 0.1-10 mL, 0.1-5 mL, 5-20 mL, 5-15 mL, or 5-10 mL.

Reservoir sample passage aperture 210 may be any size or shape that functionally transfers the appropriate quantity of sample from reservoir 200 to base 300.

In some embodiments, reservoir 200 may include volume indicators. In some embodiments, these indicators are provided on the inner or outer surface of reservoir 200 as protrusions or indentations in the material from which they are manufactured. In some embodiments, these indicators are provided as inked labels on the inner or outer surface of the reservoir 200.

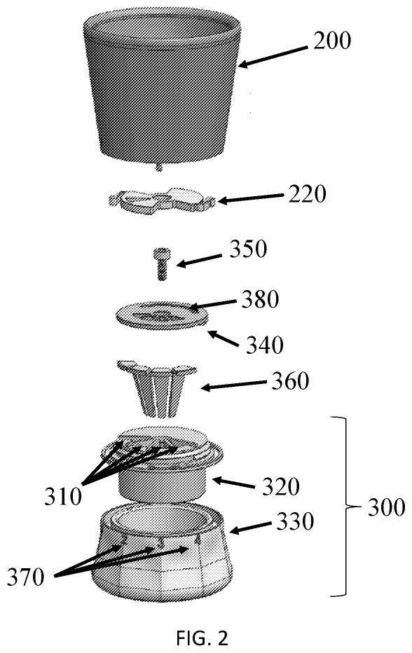

The reservoir may further comprise shutoff device 220 to control fluid connection between sample passage aperture 210 and base 300 (FIGS. 2 and 4). The shutoff device may comprise a valve or valve system (e.g., gate valve, stopcock, butterfly valve), a seal, a membrane, a hydrophilic material, or any other structure or mechanism which closes off the sample passage aperture 210. In some embodiments, shutoff device 220 comprises a mechanism to close off the sample passage aperture 210 and an opening to fluidly connect the reservoir and the one or more sample wells. In some embodiments, the opening in shutoff device 220 is the approximately the same size and shape as one or all of the sample wells. In some embodiments, the opening in shutoff device 220 is approximately the same size and shape as reservoir sample passage aperture 210.

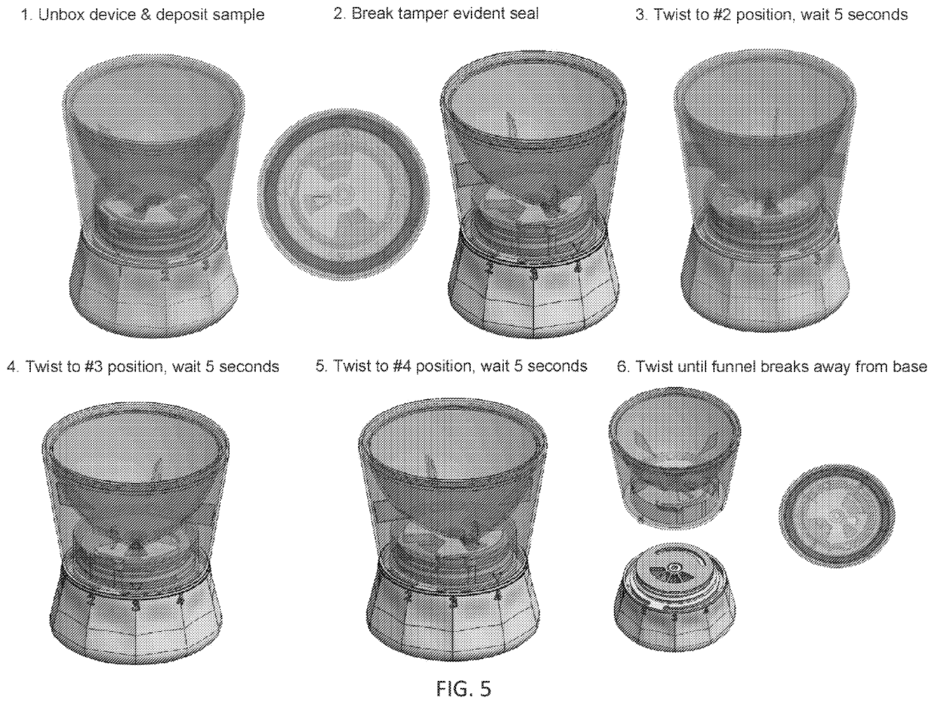

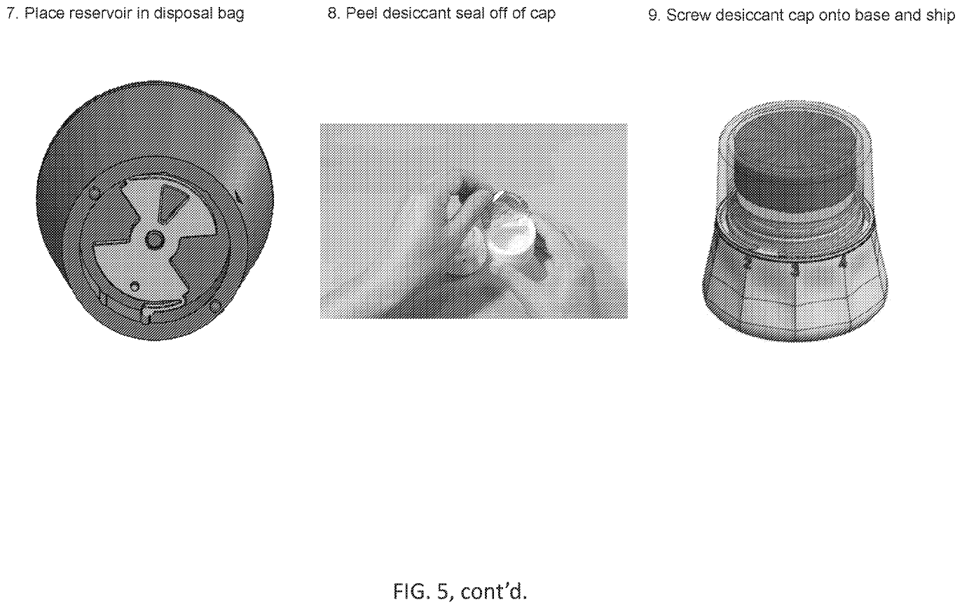

The shutoff device may be actuated manually or automatically through a metering device or valve which engages shutoff device following passage of a certain amount of time or certain amount of volume through the sample passage aperture. In some embodiments, shutoff device 220 is actuated by rotation of the reservoir (See FIG. 5, step 6). For example, when reservoir 200 is rotated with respect to shutoff device 220 to the point that no further rotation between them is possible, shutoff device 220 closes off the sample passage aperture 210 to prevent any remaining sample leaving reservoir 200.

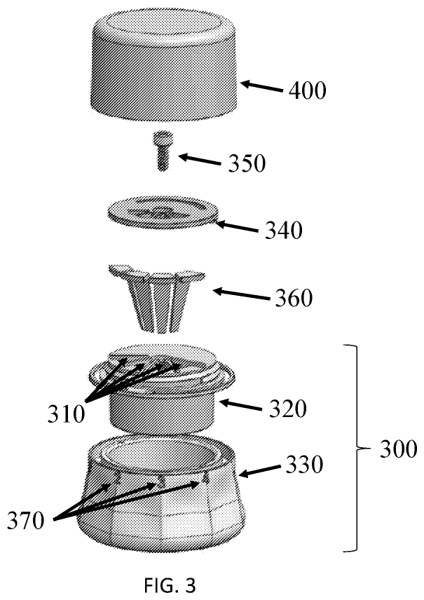

Base 300 may be a single unit or may comprise inner base 320, fully or partially inside of or contained within outer base 330 (FIGS. 2-4). Inner base 320 and outer base 330 may be integral or separate from one another. Base 300, or inner base 320, is adjustably fluidly connected to the reservoir 200 by sample passage aperture 210 in the bottom of reservoir 200.

Base 300, or inner base 320, comprises one or more sample wells 310 (FIGS. 2-4). In some embodiments, base 300, or inner base 320 comprises a single sample well. In some embodiments, base 300, or inner base 320 comprises two or more sample wells (e.g., 2, 3, 4, 5, 6, 7, 8, or more).

Sample wells 310 are capable of receiving a portion of the sample from the reservoir 200 when the sample passage aperture 210 is aligned with each sample well 310 by rotation of reservoir 200. Thus, fluid from reservoir 200 enters first sample well through sample passage aperture 210, then upon rotation of reservoir 200, the sample passage aperture 210 aligns with the second sample well for transfer of the next volume of sample until all of the sample wells have been dosed with the appropriate aliquot of sample or there is no sample remaining in reservoir 200. In some embodiments, the sample well, or the first of the one or more sample wells, is pre-aligned with the sample passage aperture 210 such that no rotation of the reservoir is needed to fill the single or first sample well.

The use of the term “sample well” does not connotate any particular size or shape of the sample receiving area. One of ordinary skill in the art can optimize various shapes and sizes of sample wells to optimize their applicability in holding the desired aliquot or quantity of sample. The sample wells may be any size, shape, or depth. In some embodiments, each of the one or more sample wells is the same size or shape. In some embodiments, at least one of the one or more sample wells may be a different size or shape.

Each of the one or more sample wells 310 comprises capture material 360 configured to capture analytes from the sample (FIGS. 2-4). In some embodiments, the capture material is the same in each of the sample wells. Alternatively, the capture material may be different in one or more of the sample wells.

In some embodiments, capture material 360 is configured for removal from sample wells 310, and recovery and analysis of captured analytes. The quantity of capture material can be optimized based on the amount of sample entering each of the one or more sample wells and the relative loading capacity of the capture material. The capture material can be sized accordingly to fit within the sample wells.

The capture material may be any matrix or structure designed to capture analytes or subgroups of analytes either specifically or non-specifically. The capture material may comprise a wide variety of adsorbent or absorbent materials. For example, the capture material may comprise modified or unmodified membranes (e.g., PVDF/nitrocellulose), chromatography resins, filter materials (e.g., filter papers or nylon filter material), cloth, cotton linter, synthetic knits (e.g., polyester hydro knit), clays, polypropylene absorbents, or combinations thereof. In some embodiments, the capture material comprises filter paper, cotton linter, or a combination thereof.

Base 300 and reservoir 200 are configured to be attachable and/or are provided to the user or proctor of the devices or systems already attached. Attaching base 300 to reservoir 200 may be facilitated through alignment or positioning of reservoir sample passage aperture 210 in regards to the first of the one or more sample wells 310. Suitable means of attaching reservoir 200 to base 300 include static and flexible or dynamic attachment means. Exemplary means of attachment in include, but are not limited to, compressive gaskets, screw mechanisms, snap couplings, compressive latching mechanisms, compressive spring mechanisms, bayonet or pin/slot couplings, ring-lock couplings, sliding mechanisms, compressive clips, and combinations thereof. In some embodiments, the reservoir comprises catches protruding from a lower face and the base comprises tabs configured to align the reservoir and the base.

In some embodiments, base 300 may further comprise retention ring 340 which functionally integrates with reservoir 200 or shutoff device 220. Retention ring may be integral to base 300 or may be separate from base 300 and attached using known fasteners, such as retention screw 350. Retention ring 340 may facilitate attachment of base 300 with reservoir 200 by proper positioning or alignment of the reservoir sample passage aperture 210 and/or reservoir shutoff device 220 with sample wells 310.

The interaction between reservoir shutoff device 220 and retention ring 340 may also facilitate sealing and detachment of reservoir 200 upon rotation past the last sample well. In some embodiments, shutoff device 220 may comprise pin 230 which functionally integrates with slot 380 in retention ring 340. Thus, when pin 230 on shutoff device 220 hits the end of slot 380 in retention ring 340 no further rotation is possible. Additional torque applied by rotating the reservoir only rotates reservoir 200 relative to shutoff device 220, which, as described above, actuates shutoff device 220. Further torque or rotation facilitates removal of reservoir 200 from base 300.

In some embodiments, retention ring 340 further comprises one or more retention ring apertures configured to align with the shutoff device opening to fluidly connect the reservoir and the one or more sample wells. Retention ring aperture(s) may be any size or shape that functionally transfers the desired sample volume. In some embodiments, the retention ring aperture is the approximately the same size and shape as one or all of the sample wells. In some embodiments, the retention ring aperture is approximately the same size and shape as the opening in the shutoff device. In some embodiments, the retention ring aperture is approximately the same size and shape as reservoir sample passage aperture 210.

In some embodiments, any or all of the aperture or openings facilitating fluid connection between reservoir 200 and base 300 may be surrounded by a seal or gasket to seal the apertures to each other or to the opposite facing structure. For example, reservoir sample passage aperture 210 may comprise an O-ring with which to form a tight seal against shutoff device 220 before, during, and after sample collection when in the positions over sample wells or in shutoff or sealed position, respectively.

In some embodiments, the outer surface of reservoir 200, base 300, and/or outer base 330 comprises indentations for handling and to aid in assistance of rotating reservoir 200. Reservoir 200, base 300, and/or outer base 330 may also comprise arrows or directional labels for reservoir 200 rotation and/or status markers 370 indicating which of sample wells 310 is aligned with sample passage aperture 210, thereby configured to receive sample from reservoir 200.

In some embodiments, the devices and systems comprise cap 400 (FIGS. 1, 3, and 4). In some embodiments, cap 400 comprises internal threads or internal teeth, similar to a screw cap, configured to rotatably mate with similar threads or teeth in base 300, inner base 320, or outer base 330, thereby hermetically sealing base 300, and coordinately sample wells 310, from outside contamination or adulteration and preventing, eliminating, and/or minimizing leakage or discharge of the contents. In some embodiments, cap 400 optionally comprises a seal 310 to further prevent unwanted leakage.

In some embodiments, the devices and systems provided herein are configured to hold and/or transport a sample, including liquid samples as described herein. In some embodiments, cap 400 further comprises desiccant 410, which when mated with base 300 is in vaporous communication with the capture material to facilitate drying of the sample on the capture material. In some embodiments, cap 400 is preloaded with desiccant 410. In some embodiments, desiccant 410 is provided separately and the user or proctor places the desiccant in the cap prior to mating with the base. In some embodiments, the desiccant is held in place in the cap with an air permeable barrier, structure, or chamber.

In some embodiments, desiccant 410 is sealed in cap 400 prior to engaging cap 400 with base 300. In some embodiments, the seal prevents attachment of cap 400 with base 300. Thus, the proctor or user of the device or system would remove seal exposing desiccant prior to attaching cap 400 to base 300. In some embodiments, a suitable seal comprises a plug, film, and/or a self-adhesive seal made of paper, wax paper, plastic materials, thin metal films, metallicized plastic, heat seals, or paper.

Exemplary desiccants include natural or synthetic desiccants including anhydrous calcium sulfate (e.g., gypsum), aluminas, including activated aluminas (e.g., aluminum oxide or Al₂O₃), glass, silicas (e.g., SiO₂, silica gels, Ascarite II® absorbents (e.g., carbon dioxide adsorbents including sodium hydroxide-coated silica), or diatomaceous silicas (e.g., Celite®, Celatom®, CAFA (Celite® Analytical Filter Aid))), a hygroscopic polymer and/or salt (e.g., CaCl₂, CaO, ZnCl₂, KOH, NaOH, CaH₂, CaSO₄, and Na₂SO₄), molecular sieves or crystalline metal aluminosilicates, activated carbon, montmorillonites or montmorillonite clays (e.g., (Al₂O₃·4SiO₂·xH₂O)), and drying agents (e.g., barium oxide, boron oxide, calcium salts (e.g., calcium chloride, calcium oxide, calcium hydride), copper(II) sulfate, lithium aluminum hydride, lithium chloride, magnesium oxide, magnesium perchlorate, magnesium sulfate, phosphorus pentoxide, potassium hydroxide, sodium, sodium hydroxide, or sodium-potassium alloy). In preferred embodiments, the desiccant is montmorillonite clay, lithium chloride, activated alumina, alkali aluminosilicate, DQ11 Briquettes, silica gel, molecular sieve, calcium sulfate, or calcium oxide. In some embodiments, the desiccant comprises a silica gel, molecular sieve, aluminosilicates, or a combination thereof.

The quantity or surface area of desiccant can be optimized to adjust the drying time of the sample. For example, to dry a larger sample or to decrease the drying time of the sample, more desiccant or increased surface area of desiccant can be used in the devices and systems. In some embodiments, the amount or surface area of desiccant is sufficient to dry the sample in less time than necessary to transport the sample. For example, the amount of desiccant dries the sample in 30 minutes, 1 hour, 2 hours, 3 hours, 4 hours, 6 hours, 12 hours, 24 hours, 36 hours, or 48 hours.

In some embodiments, cap 400 comprises an indication structure configured to produce a vibration, audible sound, and/or a haptic feedback when cap 400 is securely attached to base 300.

In some embodiments, the devices and systems comprise a tamper resistant seal or tamper evident seal to prevent tampering prior to or after collection of a sample. In some embodiments, tamper resistant seal prevents reservoir 200 from rotating to maintain proper alignment of reservoir 200 and base 300 and prevent unwanted rotation. In some embodiments, tamper evident seals prevent tampering with the reservoir 200 prior to inserting a sample. In some embodiments, the tamper resistant seal is integral to the device. Tamper resistant seals can be of various types including, but not limited to, a strap seal, foil seals, tape seals, locks, glue, epoxy, hot wax seals, and a plastic heat shrunk band. In some embodiments, the tamper resistant seal engages with the inner bottom portion of the reservoir and/or the top portion of the base to prevent unintended rotation of the reservoir.

Embodiments provide that the reservoir, base, cap, and components thereof, are manufactured using traditional manufacturing techniques known in the mechanical and manufacturing arts and provide that the reservoir, base, cap, and components thereof, are constructed from various materials. These materials can include metal, silicon, glass, ceramic, plastic, synthetic and natural polymers, rubber, or any combination thereof. In some embodiments, the reservoir, base, and/or cap are constructed from polystyrene (e.g., high impact polystyrene), high-density polyethylene, polyoxymethylene, or a polypropylene composite using similar methods known in the art of plastics construction. Other suitable materials include polytetrafluoroethylene (PTFE), nylon 6, silicone or nitrile rubbers, and butcher paper.

Methods of manufacturing can include but are not limited to milling, casting, blowing, spinning, injection molding, machining, and three-dimensional printing. In some embodiments of the present invention, the reservoir, base, and/or cap are substantially transparent so that the sample within the reservoir may be visualized by observation of the outside surface of the reservoir.

In some embodiments, the reservoir is constructed separately from the base. When constructed separately, the reservoir may be joined with the base by the manufacturer or at the point of care or prior to use. Assembly may be performed by aligning the reservoir with the base and may involve reversibly affixing the reservoir to the test chamber using interlocking surfaces (e.g., snaps), and the like.

In some embodiments, the devices and systems comprise a label or other scribe-able or scribed surface or surfaces on reservoir 200, base 300, and/or cap 400 on which to print, write, or display information. In some embodiments, a label is affixed to an outside surface of reservoir 200, base 300, and/or cap 400 by gluing, imprinting, texturing, scribing, etching, surface treating, impregnating, painting, screen printing, dyeing, coloring, embossing, or other suitable method. In some embodiments, a self-adhesive pre-printed label is affixed to the outer wall of reservoir 200, base 300, or cap 400.

In some embodiments, one or more components of the device operation are automated. For example, in some embodiments, the delivery of sample to multiple wells is automated. In some such embodiments, a sensor detects when a desired aliquot of a sample has been delivered to a first well, activating an actuator that moves one or more device components to facilitate delivery of a second aliquot to sample to a second well and/or subsequent wells.

In some embodiments, the devices and systems further comprise a sample. In some embodiments, the sample is the sample captured by the capture material.

A number of different samples are suitable with the present technology. In some embodiments, the sample is a viscous or hyper viscous sample. Exemplary viscous samples include biological fluids and samples comprising semen, synovial fluid, mucus (e.g., cervical mucus, respiratory mucus), pus, secretions (e.g., prostate secretions) and the like. Some biological fluids, such as blood, vary in viscosity and increased viscosity may be an indication of a disease, disorder, or abnormality. The devices and systems described herein would be equally applicable viscous or non-viscous samples.

In some embodiments, the sample is non-homogeneous. Non-homogeneous describes samples that are not uniform in content (e.g., homogeneous) throughout the sample, as well as samples that contain variable components, for example, components may or may not be consistently present in the sample. For example, sample contamination with other cell types or components from surrounding tissues and fluids may only be present in the sample occasionally, resulting in difficulties processing under standard conditions. The present disclosure provides methods applicable with these more complex and inconsistent samples.

Thus, in some embodiments, the devices and systems described herein assist in handling and process techniques for high viscosity, potentially complex, and/or non-homogeneous samples.

In some embodiments of the technology, the sample is a biological sample. Biological specimens include but are not limited to a sample from a subject such as an animal (e.g., a mammal (e.g., a primate (e.g., human))). A sample from a subject can be of any appropriate type, such as a sample of fluid, tissue, organ, or a combination thereof. Exemplary biological samples, include but are not limited to fluid samples (e.g., urine, blood, serum, saliva, semen, secretions (e.g., vaginal secretions), central nervous system fluids, lavages, and the like). In some embodiments, the sample is semen. Any of the biological samples described herein may be obtained from the subject using any known technique. The biological sample can also be a sample of other biological material, such as food, including food materials derived from plants or animals or combinations thereof.

In some embodiments, reservoir 200, sample wells 310, or capture material 360 includes reagents for use in processing the sample. In some embodiments, a reagent is used to process a sample, e.g., to digest a solid sample with appropriate reagents (e.g., chemicals, acids, bases, and/or enzymes (e.g., proteases)). In some embodiments, reagents are used to extract or partially purify an analyte from a sample. In some embodiments, reagents are used to stabilize an analyte in a sample.

In some embodiments, the sample is in the sample wells. In some embodiments, the sample is partially or completely dried on the capture materials.

### 3) Methods of Use

The disclosure provides methods for using the devices or systems herein to collect a sample. In some embodiments, the methods comprise providing the device or system, providing a sample in the reservoir, rotating the reservoir to fill each of the one or more sample wells, removing the reservoir, and capping the base (e.g., sealing the base with a cap). In some embodiments, the methods further comprise any one of, any combination of two or more of, or all of; breaking the tamper resistant seal, preparing the reservoir for disposal, removing seal from cap, affixing a label to the device, and preparing shipping container.

The disclosure also provides methods for using the devices or systems herein to store and/or transport a sample. The methods comprise collecting a sample with the disclosed devices and systems; removing the reservoir; and capping or sealing the base with the cap. Once the base is sealed with the cap, the desiccant is in vaporous communication with the capture material and facilitates drying of the sample on the capture material.

The disclosure further provides methods for storing a semen sample comprising: collecting a semen sample in a device comprising a plurality of regions (e.g., two, three, four, or more) each having a capture material such that a portion of the semen sample is retained by the capture material in said plurality of regions; and drying the semen sample. The capture material may be the same or different in any one or all of the plurality of regions. Exemplary capture materials are provided elsewhere herein.

In some embodiments, the methods further comprise sealing the device to enclose the semen sample. In some embodiments, at least a portion of the drying step occurs following the sealing.

In some embodiments, the methods further comprise any or all of; preparing the reservoir for disposal, removing seal from cap, affixing a label to the device, and preparing shipping container. In some embodiments, the methods further comprise shipping the device to a testing location.

In some embodiments, the methods further comprise analyzing the sample (e.g., the semen sample) after it is dried. The analysis can include any assay necessary to characterize the sample. In some embodiments, the analysis comprises conducting a biological assay with sample from obtained from capture material from one or more or all of the regions (e.g., sample wells). In some embodiments, the biological assay detects the presence or absence of a biomarker (e.g., a cancer biomarker, e.g., a prostate cancer biomarker).

In some embodiments, the methods further comprise rehydrating the sample. In some embodiments, the rehydrating comprising incubating the capture material with a buffer or solvent at a defined temperature about 4° C. to about 90° C. for a period of time and separating the capture material from the rehydrated sample. In some embodiments, the incubating is carried out at about 20° C. to about 70° C. In select embodiments, the incubating is carried out at about 4° C., about 10° C., about 15° C., about 20° C., about 25° C., about 30° C., about 35° C., about 40° C., about 45° C., about 50° C., about 55° C., about 60° C., about 65° C., about 70° C., about 75° C., or about 80° C.

The incubation may be carried out and optimized for the time necessary to allow at least partial rehydration of the sample. The total time is dependent on type and quantity of the sample, type and quantity of the capture material, and solvent or buffer. In some embodiments, the incubation time is 1-60 minutes (e.g., 5-50 minutes, 10-40 minutes, 20-30 minutes). In some embodiments, the incubation time is about 1 min, about 5 min, about 10 min, about 15 min, about 20 min, about 25 min, about 30 min, about 35 min, about 40 min, about 45 min, about 50 min, about 55 min, or about 60 min.

The rehydration buffer or solvent may be any of those that allow rehydration and are compatible with subsequent downstream processing (e.g., detecting and/or measuring any of the disclosed biomarkers). Exemplary buffers and solvents suitable for use with the methods disclosed herein include, but are not limited to, water, isotonic saline, phosphate buffered saline (PBS), 4-2-hydroxyethyl-1-piperazineethanesulfonic acid buffer (HEPES), 3-(N-morpholino)propanesulfonic acid buffer (MOPS), and piperazine-N,N′-bis(2-ethanesulfonic acid) buffer (PIPES), Tris buffered saline (TBS), tromethamine hydrochloride, Hanks' balanced salt solution, Earle's balanced salt solution, and Ringer's solution.

Rehydrating the samples as described herein results in more homogeneous samples compared to the same samples recovered by centrifugation, thereby allowing consistent processing of more complex, viscous samples not previously achievable.

The rehydrated sample may be separated from the capture material using methods known in the art (e.g., centrifugation, magnetic separation, immobilization etc.). The rehydrated sample may be used directly or following a pretreatment step to modify the character of the sample. Such pretreatment step may include, for example, filtration, precipitation, dilution, distillation, mixing, concentration, inactivation or removal of interfering components, the addition of reagents, lysing, and the like.

As such, the disclosure additionally provides methods for collection and testing of a semen sample comprising: collecting a semen sample on a plurality of capture materials; drying the semen sample; storing and/or shipping dried semen samples; and conducting a biological assay on sample obtained from one or more of the plurality of capture materials.

FIG. 5 illustrates an exemplary methodology of using the disclosed devices and systems. In this method, the user or proctor prepares the device or system for the sample by removing the device or system from the packaging. Following collection of the sample, the tamper evident seal is broken and the reservoir is successively rotated to each sample well, with a hold period (e.g., 5 seconds) before each successive rotation. After the final sample well is filled, further rotation seals the reservoir and removes it from the base. The reservoir is placed in a container for disposal. The seal is removed from the cap and the cap is attached to the base and prepared for storage and shipment.

The time period for which the sample is stored may be at least the time period needed for the device to be transported from the site of use, e.g., an external location, to the site of analysis (e.g., testing location) of the sample. For example, the sample may be stored for a period of several minutes, hours, days, months, or more.

In some embodiments, the storing is carried out using the same device as the drying, collecting, and shipping. In some embodiments, the samples are stored at ambient or room temperature (e.g., 15-30° C.). In some embodiments, the sample are stored at cool temperatures, below that of ambient or room temperature (e.g., 2-15° C., −20° C. or lower).

Drying the sample may confer increased stability for storage. In some embodiments, the sample when dried is stable for up to 7 days at elevated temperatures above room temperature (e.g., 50-60° C.). In some embodiments, the dried sample is stable for over 1 week, over 1 month, over 6 months, over 1 year when frozen.

The device or systems may be used to store or transport a sample under any temperature conditions. In some embodiments, the samples are transported or stored at ambient or room temperature (e.g., 15-30° C.). In some embodiments, the sample are transported or stored at cool temperatures, below that of ambient or room temperature (e.g., 2-15° C.).

### 4) Kits

The disclosure also provides kits comprising the devices or systems disclosed herein.

The kits may additionally comprise one or more of: peel and stick labels, disposal containers, shipping containers and labels, and fillable document(s) with information regarding user details.

Individual member components of the kits may be physically packaged together or separately. For example, the base and the reservoir may be packaged together as a single device and the cap may be packaged together with the device or separately.

The kits can also comprise instructions for using the components of the kit. The instructions are relevant materials or methodologies pertaining to the kit. The materials may include any combination of the following: background information, list of components, brief or detailed methods for using the system, trouble-shooting, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods.

All references, including publications, patent applications, and patents, cited herein are hereby incorporated by reference to the same extent as if each reference were individually and specifically indicated to be incorporated by reference and were set forth in its entirety herein.

We claim:

1. A device comprising:
a reservoir configured for collecting and holding a sample; and
a base comprising one or more sample wells, each comprising a capture material,
wherein the reservoir is adjustably, fluidly connected each of the one or more sample wells.

2. The device of claim 1, wherein the reservoir is detachably connected to the base.

3. The device of claim 1 or 2, wherein the reservoir comprises catches protruding from a lower face and the base comprises tabs configured to align the reservoir and the base.

4. The device of any of claims 1-3, wherein a sample passage aperture in a bottom wall of the reservoir is configured to align with the one or more sample wells in the base at a starting position or upon rotation of the reservoir.

5. The device of any of claims 1-4, wherein the reservoir comprises an inner surface which is tapered to the sample passage aperture.

6. The device of any of claims 1-5, wherein the reservoir further comprises a shutoff device to control fluid connection between the sample passage aperture and the sample wells.

7. The device of claim 6, wherein the shutoff device comprises an opening to fluidly connect the reservoir and the one or more sample wells.

8. The device of claim 6 or 7, wherein the base further comprises a retention ring which functionally integrates with the shutoff device.

9. The device of claim 8, wherein the shutoff device comprises a pin which functionally integrates with a slot in the retention ring facilitating rotation and removal of the reservoir from the base.

10. The device of claim 8 or 9, wherein the retention ring comprises one or more retention ring apertures configured to align with the shutoff device opening to fluidly connect the reservoir and the one or more sample wells.

11. The device of any of claim 1-10, wherein the capture material comprises filter paper, cotton linter, or a combination thereof.

12. The device of any of claims 1-11, further comprising a cap comprising desiccant.

13. The device of claim 12, wherein the cap is configured to attach to the base following detachment of reservoir.

14. The device of claim 12 or 13, wherein the cap is a screw cap.

15. The device of any of claims 12-14, wherein the desiccant is selected from silica gel, molecular sieve, an aluminosilicate, or a combination thereof.

16. The device of any of claims 1-15, further comprising a tamper resistant seal.

17. The device of any of claims 1-16, further comprising a sample.

18. The device of claim 17, wherein the sample comprises a biological fluid.

19. The device of claim 17 or 18, wherein the sample comprises semen.

20. The device any of claims 17-19, wherein the sample is in the one or more sample wells.

21. The device of any of claims 17-20, wherein the sample is dried on the capture material.

22. A system comprising
a reservoir configured for collecting and holding a sample;
a base comprising one or more sample wells, wherein each sample well comprises a capture material; and
a cap comprising a desiccant.

23. The system of claim 22, wherein the base is configured to independently attach to the reservoir and the cap.

24. The system of claim 22 or 23, wherein the reservoir comprises catches protruding from a lower face and the base comprises tabs configured to align the reservoir and the base.

25. The system of any of claims 22-24, wherein a sample passage aperture in a bottom wall of the reservoir is configured to align with the one or more sample wells in the base at a starting position or upon rotation of the reservoir.

26. The system of any of claims 22-25, wherein the reservoir comprises an inner surface which is tapered to the sample passage aperture.

27. The system of any of claims 22-26, wherein the reservoir further comprises a shutoff device to control fluid connection between the sample passage aperture and the sample wells.

28. The system of claim 27, wherein the shutoff device comprises an opening to fluidly connect the reservoir and the one or more sample wells.

29. The system of any of claims 22-28, wherein the base further comprises a retention ring which functionally integrates with the shutoff device.

30. The system of claim 29, wherein the shutoff device comprises a pin which functionally integrates with a slot in the retention ring facilitating rotation and removal of the reservoir from the base.

31. The system of claim 29 or 30, wherein the retention ring comprises one or more retention ring apertures configured to align with the shutoff device opening to fluidly connect the reservoir and the one or more sample wells.

32. The system of any of claims 22-31, wherein the cap is a screw cap.

33. The system of any of claims 22-32, wherein the desiccant is selected from silica gel, molecular sieve, an aluminosilicate, or a combination thereof.

34. The system of any of claims 22-33, wherein the capture material comprises filter paper, cotton linter, or a combination thereof.

35. The system of any of claims 22-34, further comprising a sample.

36. The system of claim 34, wherein the sample comprises a biological fluid.

37. The system of claim 35 or 36, wherein the sample comprises semen.

38. The system of any of claims 35-37, wherein the sample is in the one or more sample wells.

39. The system of any of claims 35-38, wherein the sample is dried on the capture material.

40. A method for collecting a sample, comprising:
providing a device of any of claims 1-21 or a system of any of claims 22-39;
providing a sample in the reservoir;
rotating the reservoir to fill each of the one or more sample wells;
removing the reservoir; and
capping the base.

41. The method of claim 40, further comprising breaking the tamper resistant seal.

42. The method of claim 40 or 41, further comprising removing seal from cap prior to capping the base.

43. The method of any of claims 40-42, wherein the sample comprises a biological fluid.

44. The method of any of claims 40-43, wherein the sample comprises semen.

45. A method of storing and/or transporting a sample, comprising:
collecting a sample in a device of any of claims 1-21 or a system of any of claims 22-39;
removing the reservoir; and
capping the base.

46. The method of claim 45, wherein the sample comprises a biological fluid.

47. The method of claim 45 or 46, wherein the sample comprises semen.

48. Use of a device of any of claims 1-21 or a system of any of claims 22-39 for collection, storage, or transport of a sample.

49. A kit comprising:
a device of any of claims 1-21 or a system of any of claims 22-39; and
one or more of;
a disposal bag;
a shipping container; and
instructions.

50. A method for storing a semen sample, comprising:
collecting a semen sample in a device comprising a plurality of regions each having a capture material such that a portion of the semen sample is retained by the capture material in the plurality of regions; and
drying the semen sample.

51. The method of claim 50, further comprising the step of sealing the device to enclose the semen sample.

52. The method of claim 51, wherein at least a portion of the drying step occurs following the sealing.

53. The method of any of claims 50-52, further comprising the step of shipping the device to a testing location.

54. The method of any of claims 50-53, further comprising the step of analyzing the semen sample after the drying step.

55. The method of claim 54, wherein said analyzing comprises conducting a biological assay with sample obtained from capture material from one or more of the plurality of regions.

56. The method of claim 55, wherein the biological assay detects the presence or absence of a biomarker.

57. The method of any of claims 50-56, wherein the plurality of regions comprises three or more regions.

58. The method of any of claims 50-57, wherein the plurality of regions comprises four or more regions.

59. A method for collection and testing of a semen sample, comprising:
collecting a semen sample on a plurality of capture materials;
drying the semen sample;
storing and/or shipping dried semen samples; and
conducting a biological assay on sample obtained from one or more of the plurality of capture materials.

60. The method of claim 59, wherein the biological assay detects the presence or absence of a biomarker.
